(19) United States
(12) Patent Application Publication
(10) Pub. No.: US 2024/0247410 A1
(43) Pub. Date: Jul. 25, 2024

(54) METHOD OF GENERATING MULTIFUNCTIONAL MYCELIUM PRODUCTS

(71) Applicant: Mushrooms Inc., Estero, FL (US)

(72) Inventors: Kimberly Zawi Carlson, Estero, FL (US); Donald Steinberg, Estero, FL (US); Hyder Ali Khoja, New Westminster (CA); Maqsad Suriev, Burnaby (CA)

(73) Assignee: Mushrooms Inc., Estero, FL (US)

(21) Appl. No.: 18/488,639

(22) Filed: Oct. 17, 2023

Related U.S. Application Data
(60) Provisional application No. 63/371,683, filed on Aug. 17, 2022.

(51) Int. Cl. D01F 2/00; A01G 18/20; A01G 18/50; A01G 18/64; A01G 18/69; A01G 18/70; A01G 18/80; A61K 47/46; C12Q 1/40; D01D 1/10; D01D 5/06

(52) U.S. Cl. CPC D01F 2/00; A01G 18/80; A61K 47/46; C12Q 1/40; D01D 1/103; D01D 1/106; D01D 5/06; A01G 18/20; A01G 18/50; A01G 18/64; A01G 18/69; A01G 18/70; D10B 2201/20; D10B 2401/13; D10B 2501/00; D10B 2509/00

(57) ABSTRACT

A method of generating multifunctional mycelium products (thread, foam, films, gels and liquids) derived from biodegraded waste and fungi includes inoculating a carbon-containing substrate with fungi; growing the fungi to form a mature mycelium; separating the mycelium from the carbon-containing substrate; and drying the mature mycelium. The mycelium is either injected into a mold and expanded, fermented to produce a liquid, or blended with water and gelling agents to form a hydrogel. The products sequester carbon and inherently combat pathogens, notably MRSA. Further augmented with synthetic enzyme substrates, the products offer bacterial detection. A standout feature is their capacity to hold and release therapeutic agents, diversifying their use from sustainable textiles to skincare, wound healing, and precise drug delivery.

### CROSS-REFERENCE TO RELATED APPLICATION

This application claims the benefit of priority of U.S. provisional application No. 63/371,683, filed Aug. 17, 2022, the contents of which are herein incorporated by reference.

### BACKGROUND OF THE INVENTION

The present invention relates to methods of producing products for bacterial detection, bacterial growth inhibition, and nutrient delivery and, more particularly, to a method of generating multifunctional mycelium products, and the products made thereby, such as thread, foam, films, gels, and liquids.

Bacteria (e.g., Escherichia coli [e-coli], salmonella, methicillin-resistant Staphylococcus aureus [MRSA], Listeria) are found in critical environments like medical facilities. Timely detection of these bacteria is of paramount importance for effective intervention, especially in high-risk settings.

The alarming surge in bacterial infections, particularly by MRSA—a strain defiant against many existing treatments, culminated in 20,000 U.S. fatalities in 2019. The absence of a direct pharmaceutical countermeasure for MRSA, combined with the challenges of traditional bactericidal products, emphasizes the urgent need for timely bacterial detection and effective intervention, especially in high-risk settings.

Alongside the challenge of bacterial detection, there's an increasing need for innovative mechanisms to deliver essential nutrients in a controlled manner, whether for medical, wellness, or cosmetic purposes.

As can be seen, there is a need for a sustainable alternative to bactericidal products that can reliably detect bacterial presence, disease causing thresh hold and potentially prevent further spread, facilitating timely medical intervention, and delivering vital nutrients for enhanced well-being.

### SUMMARY OF THE INVENTION

In one aspect of the present invention, a method of formulating and manufacturing a mycelium-based product, comprises inoculating a carbon-containing substrate with fungi; growing the fungi to form a mature mycelium; separating the mature mycelium from the carbon-containing substrate; drying the mature mycelium; injecting the mature mycelium into a mold; and expanding the mature mycelium into a foam by reducing a pressure in the mold and/or increasing a temperature within the mold.

In another aspect of the present invention, a method of manufacturing a mycelium-based product comprises fermenting the mature mycelium to produce a mycelium liquid.

In yet another aspect of the present invention, a method of manufacturing a mycelium-based product comprises blending the mature mycelium with water and gelling agents to form a mycelial hydrogel.

Mycelium products inherently possess the capability for inhibiting bacterial growth, including formidable antibiotic-resistant strains like MRSA, making them apt for biomedical, military, aerospace, and lifestyle uses. The products find relevance in a wide variety of fields, including but not limited to healthcare, for timely identification of pathogens, enhancing wound healing, and nutrient delivery; home comforts; and environmental filters. Mycelium-based threads may be woven into sports gear, medical textiles, therapeutic garments, and automotive interiors tailored to a multitude of health needs, such as bolstering immunity. Furthermore, their intrinsic radiation shielding capabilities render them invaluable in aerospace and military sectors. Unlike conventional methods that generate textile waste and pose environmental threats, the method disclosed herein emphasizes eco-friendliness, including carbon sequestration, standing as a beacon of sustainability, efficiency, and multifunctionality, resonating with global trends.

These and other features, aspects and advantages of the present invention will become better understood with reference to the following drawings, description, and claims.

### BRIEF DESCRIPTION OF THE DRAWINGS

FIG. 1 is a schematic view of a system for generating mycelium-derived thread;

FIG. 2 is a schematic view of the enzyme substrate-loaded thread generated thereby to detect a bacterial presence;

FIG. 3 is a flow chart of a process of fabricating mycelium-derived thread;

FIG. 4 is a schematic view of a product made therefrom; and

FIG. 5 is a schematic view of transdermal delivery of a substance from the product of FIG. 4.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a mycelium-based product and a method of generation thereof. Mycelium may be used to produce products including but not limited to fabrics, thread, casted forms, liquid sprays, and gels.

The present method harnesses biodegraded waste and fungi to produce mycelium products that inhibit bacterial growth, deliver essential nutrients, and detect bacterial presence. A multifaceted or multifunctional mycelium thread produced thereby, enhanced with synthetic enzyme substrates, offers antimicrobial defense, bacterial detection, and a novel nutrient delivery system. These products provide invaluable microbial detection, a feature especially pivotal in high-risk environments such as hospitals and vital in medical scenarios for timely intervention.

The production method is adaptable to varying factors such as the specific fungus strain, the intended final product, and individual circumstances. This method may be effectively streamlined through automation or computer-controlled methodologies, ensuring efficient and consistent production at scale.

Carbon-rich waste materials, such as hemp and other plant matter, may be inoculated with and biodegraded by fungi. The growth may occur in a sterilized bioreactor, ensuring contaminant-free fungal growth. The growing mycelium forms a hyphal network, primarily structured with chitin, a prevalent polysaccharide second only to cellulose. Once matured, the mycelium is harvested by filtration, separating it from its substrate. The harvested mycelium may then be dried.

Mycelium self-digests by autolysis, activating chitinases that break down chitin into smaller molecules, leading to chitosan production, which possesses antimicrobial, antioxidant, and antitumor capabilities, including synergistic bacteriostatic and bactericidal qualities. The resultant chitosan, superior to chitin in properties like solubility and biocompatibility, may be extracted through filtration, purification, and drying.

As the cultivated mycelium proliferates and establishes a network of interconnected hyphae, it forms a three-dimensional structure that lends itself to the production of foam. It may be mixed with additives like natural binders or foaming agents and placed in molds or containers of varying shapes and sizes, depending on the intended application of the foam. The mixture may be expanded by adjusting environmental conditions. The resultant foam is lightweight. Mycelium foams have structural integrity suitable for use as eco-friendly insulation in construction and soil remediation and as they are biodegradable, mycelium foams stand out as prime candidates for eco-friendly cushioning in packaging. The foams provide antifungal benefits when integrated into footwear as padding or insoles.

Mycelium liquids may be produced by fermentation of the cultivated mycelium in a bioreactor, releasing metabolites into the liquid medium. The resulting mycelium liquid is rich in bioactive compounds. In the realm of skincare, mycelium liquids can be blended into products for their therapeutic benefits or applied directly for wound healing. They may be used as a spray or mixed into formulations like lotions or serums to harness mycelium's natural benefits.

Mycelium gels may be produced by blending a mycelium-rich mass with a solution containing water and gelling agents. The resulting hydrogel retains moisture. Mycelium gels are versatile and are suitable as a base for drug delivery in topical treatments, direct application to wounds or burns, and integration into cosmetics for enhanced skin rejuvenal benefits. These products possess the unique ability to act as a delivery conduit for added agents through transdermal delivery. Agents for transdermal delivery may be added at different production stages for customization of desired results.

Mycelium films may be produced by spin-coating the mycelial hydrogel. Adjusting the rotation speeds of spinning plates determines the film thickness. Post-spin coating, the films are dried and maintain antimicrobial and any microbial detection properties. Mycelium films may serve as sustainable packaging solutions, and in medical settings, they can act as protective barriers or wraps, such as wound dressings. They may also find utility in the cosmetic sector as a base for patches or masks for skincare and/or drug delivery.

A monofilament yarn may be produced by a) wet spinning a hydrogel mass prepared from only the intermediate product containing chitosan, or b) mixture of the intermediate product with a minimal amount of cellulose xanthate, an intermediate product in the viscose method of rayon production. The resulting mass may be de-aerated, filtered, and extruded through spinnerets. The monofilaments from a single spinneret may be combined into a single rayon thread. For medical applications, the mycelium thread may be woven or stitched into wound dressings, sutures, or medical textiles. In lifestyle contexts, the thread is ideal for incorporation into clothing, accessories, or home textiles due to its antimicrobial properties. Athletes may benefit by integrating the mycelium threads into athletic wear or equipment for its therapeutic and protective attributes.

The spinnerets are not particularly limited and may be manufactured, for example, of platinum. The spinnerets may have a range of hole sizes and the number of holes may vary. For example, spinnerets may contain about 10 to 120 holes to produce Rayon and may have as many as about 3000 holes to produce tire cord. The holes may generally have a diameter from about 0.0002 inches to about 0.0004 inches.

One of its standout attributes is the mycelium products' ability to host and gradually release beneficial agents, such as nutrients, therapeutic agents, or specific active compounds. Nutrients include, for example, proteins, fats, carbohydrates, vitamins, and minerals. The production method ensures precise application of nutrients to specific areas, potentially heightening effectiveness and sometimes resulting in superior absorption. This feature ensures continuous nourishment, offers targeted therapeutic benefits, and signifies a paradigm shift in how we perceive and use textiles. Unlike conventional methods demanding frequent re-application or ingestion, the mycelium-derived products of the present subject matter can steadily release nutrients for extended durations, promising sustained advantages. The controlled nutrient release ensures safety by mitigating overconsumption risks. This approach also caters to individuals facing challenges with oral supplement ingestion. Enriching everyday items with nutrients elevates their intrinsic value, transforming them into tools that bolster health.

### Additives for Nutrient Delivery in Mycelium Production

In the process of cultivating the mycelium thread, various additives can be introduced at distinct stages of production to enhance the functional properties of the end product. The incorporation of these additives is meticulously timed based on their sensitivity to factors such as heat, ensuring optimal retention of their beneficial attributes. Examples of such additives include, but are not limited to, lavender oil, known for its calming properties; cannabidiol (CBD), recognized for its potential anti-inflammatory effects; Ayurvedic formulations, which have been used traditionally for holistic wellness; probiotics, which can aid in skin health; essential vitamins and minerals; active compound extracts from medicinal plants; creatine, which is vital for muscle energy; taurine, known for its potential to aid in cardiovascular function; and proteins, which can provide support for tissue repair and regeneration. Additionally, considering the broader medical applications, additives may include but are not limited to antimicrobial agents, analgesics, and other therapeutic compounds. These additives serve as illustrative examples. The method is amenable to the inclusion of a diverse range of nutrients and compounds depending upon the selected functionality of the mycelium thread.

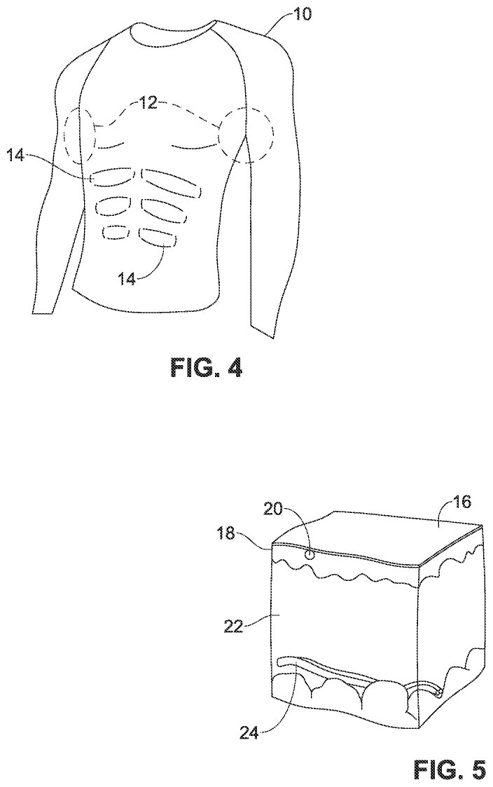

In some embodiments, the mycelium-derived product may comprise heat-stable healing elements such as herbal extracts and vitamins or heat-sensitive components like probiotics. FIG. 4 shows a garment 10 exemplifying a use of the mycelium threads to form antibacterial regions 12 and nutrient delivery regions 14.

FIG. 5 illustrates transdermal delivery of nutrients 20 released from a mycelium-derived product through a user's epidermis 18 and dermis 22. The nutrients 20 may enter the user's blood circulation via blood vessels 24.

By integrating enzyme-specific fluorogenic substrates or chromogenic substrates into the mycelium, the mycelium-based product achieves a monumental leap in real-time bacterial detection by exhibiting a color change or emitting light in the presence of bacteria. The method's capability to manufacture products that detect bacterial presence offers a pivotal advantage, especially in the face of escalating bacterial resistance, which is a considerable global health concern. Bacterial detection substrates may be added at different production stages for customization of desired results.

### Examples of Reactive Substrates for Bacterial Detection

In the context of bacterial detection, various substrates can be utilized in conjunction with the mycelium-derived products of the present subject matter to detect specific bacterial strains. For instance, when aiming to detect strains of E. coli, substrates such as 4-Nitrophenyl-β-D-glucuronide and 5-Bromo-4-chloro-3-indolyl-β-D-galactopyranoside can be employed. It is noteworthy that enzymes like β-galactosidase and β-glucuronidase are commonly produced by E. coli strains. Specifically, β-galactosidase is produced by over 90% of E. coli strains, encompassing Enterohemorrhagic E. coli (EHEC).

For the detection of S. aureus, the substrate 4-Methylumbelliferyl α-D-glucopyranoside (often referred to as MUD) is particularly significant. MUD is the fluorogenic substrate of the alpha-glucosidase enzyme. Upon interaction, MUD undergoes a reaction that results in the product 4-Methylumbelliferone (4MU), which emits a peak of 440 nm in the fluorescence spectra. This substrate has been extensively researched for its effectiveness in identifying methicillin-resistant S. aureus (MRSA). The enzyme α-glucosidase acts as an identifier enzyme (ID marker) for S. aureus, helping in distinguishing S. aureus from other Staphylococci. The substrates disclosed herein serve as non-exhaustive examples and are not limiting. The mycelium-derived products can work synergistically with a range of other substrates for bacterial detection.

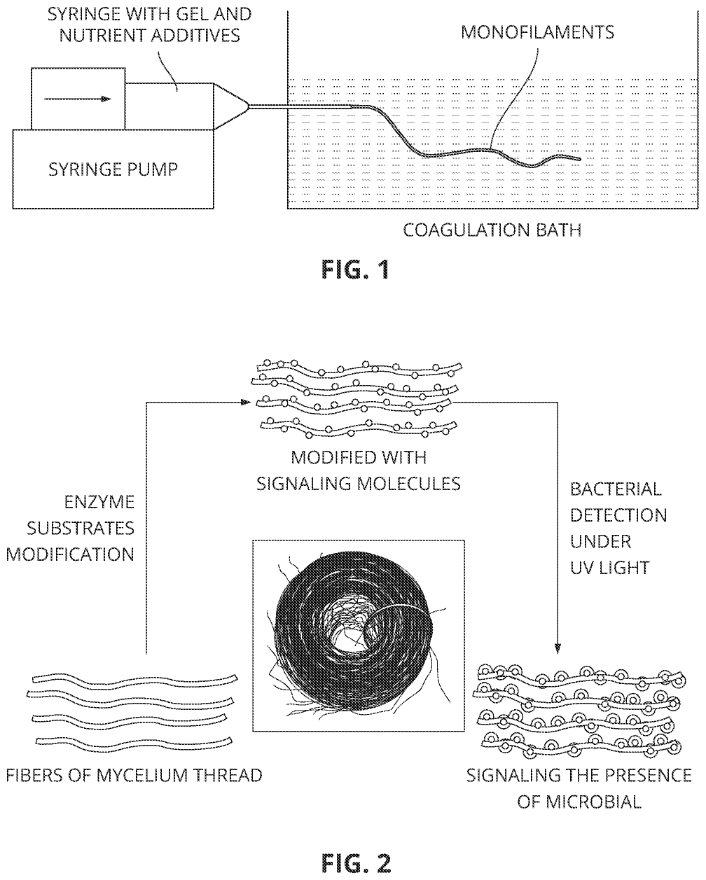

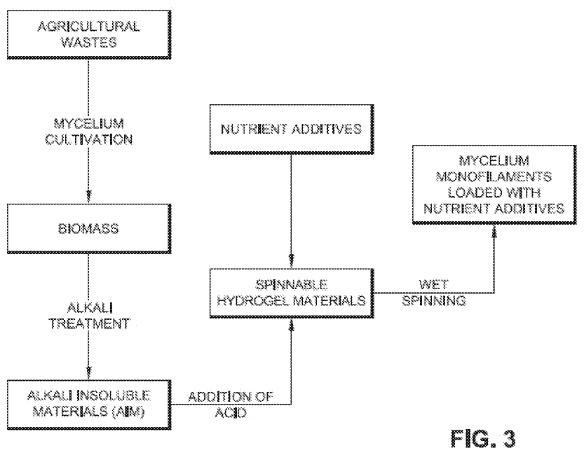

Referring to FIGS. 1 through 5, FIG. 1 illustrates extrusion of mycelium-containing cellulose xanthate gel according to an embodiment of the present invention. The mycelium-containing cellulose xanthate gel, which may also contain nutrient additives, is pumped from a syringe into a coagulation bath to form mycelium-containing monofilaments. As FIG. 3 illustrates, the mycelium may be cultivated on agricultural waste to produce a mycelium-containing biomass. The biomass may be exposed to alkali treatment, which dissolves all but alkali-insoluble materials (AIM). An acid is added to the AIM and the acid-treated AIM is mixed with nutrient additives into spinnable hydrogel materials. The hydrogel is wet spun to form mycelium monofilaments loaded with nutrient additives.

As shown in FIG. 2, the resulting mycelium thread fibers may have a fluorogenic substrate which signal the presence of bacteria due to degradation by bacterial enzymes to produce fluorescent signaling molecules, which fluoresce when exposed to ultraviolet light. FIGS. 4 and 5 are discussed supra.

It should be understood, of course, that the foregoing relates to exemplary embodiments of the invention and that modifications may be made without departing from the spirit and scope of the invention as set forth in the following claims.

### LIST OF REFERENCES

1. ADDIN ZOTERO BIBL {“uncited”:[ ],“omitted”:[ ],“custom”:[ ]} CSL_BIBLIOGRAPHY Antinori, M. E., Contardi, M., Suarato, G., Armirotti, A., Bertorelli, R., Mancini, G., Debellis, D., & Athanassiou, A. (2021). Advanced mycelium materials as potential self-growing biomedical scaffolds. Scientific Reports, 11(1), Article 1. https://doi.org/10.1038/s41598-021-91572-x
2. CDC. (2019, March 5). Staph infections can kill. Centers for Disease Control and Prevention. https://www.cdc.gov/vitalsigns/staph
3. Fach, P., Perelle, S., Dilasser, F., Grout, J., Dargaignaratz, C., Botella, L., Gourreau, J.-M., Carlin, F., Popoff, M. R., & Broussolle, V. (2002). Detection by PCR-Enzyme-Linked Immunosorbent Assay of Clostridium botulinum in Fish and Environmental Samples from a Coastal Area in Northern France. Applied and Environmental Microbiology, 68(12), 5870-5876. https://doi.org/10.1128/AEM.68.12.5870-5876.2002
4. Li, H., & Zhu, J. (2018). Differentiating Antibiotic-Resistant Staphylococcus aureus Using Secondary Electrospray Ionization Tandem Mass Spectrometry. Analytical Chemistry, 90(20), 12108-12115. https://doi.org/10.1021/acs.analchem.8b03029
5. Mirian, A., Bosch, A., Lasch, P., Stämmler, M., Serra, D. O., Degrossi, J., Gatti, B., Vay, C., D'aquino, M., Yantorno, O., & Naumann, D. (2009). Rapid identification of Burkholderia cepacia complex species including strains of the novel Taxon K, recovered from cystic fibrosis patients by intact cell MALDI-ToF mass spectrometry. Analyst, 134(6), 1138-1148. https://doi.org/10.1039/B822669E
6. Pescaroli, G., & Alexander, D. (2015). A definition of cascading disasters and cascading effects: Going beyond the “toppling dominos” metaphor. Undefined. https://www.semanticscholar.org/paper/A-definition-of-cascading-disasters-and-cascading-Pescaroli-Alexander/06074f128dfc90ea9adb5d54b629bcc586199089
7. Sadat Ebrahimi, M. M., & Schönherr, H. (2014). Enzyme-Sensing Chitosan Hydrogels.Langmuir, 30(26), 7842-7850. https://doi.org/10.1021/1a501482u
8. Suaifan, G. A. R. Y., Alhogail, S., & Zourob, M. (2017). Rapid and low-cost biosensor for the detection of Staphylococcus aureus. Biosensors & Bioelectronics, 90, 230-237. https://doi.org/10.1016/j.bios.2016.11.047
9. Svensson, S. E., Ferreira, J. A., Hakkarainen, M., Adolfsson, K. H., & Zamani, A. (2021). Fungal textiles: Wet spinning of fungal microfibers to produce monofilament yarns. Sustainable Materials and Technologies, 28, e00256. https://doi.org/10.1016/j.susmat.2021.e00256

What is claimed is:

1. A method of manufacturing a mycelium-based product, comprising:
inoculating a carbon-containing substrate with fungi;
growing the fungi to form a mature mycelium; and
separating the mature mycelium from the carbon-containing substrate;
drying the mature mycelium;
injecting the mature mycelium into a mold; and
expanding the mature mycelium into a foam by reducing a pressure in the mold and/or increasing a temperature within the mold.

2. A method of manufacturing a mycelium-based product, comprising:
inoculating a carbon-containing substrate with fungi;
growing the fungi to form a mature mycelium;
separating the mature mycelium from the carbon-containing substrate drying the mature mycelium; and
fermenting the mature mycelium to produce a mycelium liquid.

3. The method of claim 2, further comprising:
blending the mycelium liquid into a skincare formulation.

4. A method of manufacturing a mycelium-based product, comprising:
inoculating a carbon-containing substrate with fungi;
growing the fungi to form a mature mycelium;
separating the mature mycelium from the carbon-containing substrate drying the mature mycelium; and
blending the mature mycelium with water and gelling agents to form a mycelial hydrogel.

5. The method of claim 4, further comprising:
spin-coating the mycelial hydrogel onto a spinning plate having a predetermined rotational speed to form a film; and
drying the film.

6. The method of claim 4, further comprising:
de-aerating the mycelial hydrogel;
filtering the mycelial hydrogel; and
extruding the mycelial hydrogel through spinnerets to form monofilaments.

7. The method of claim 4, further comprising:
blending the mycelial hydrogel with cellulose xanthate to produce a blended hydrogel; and
extruding the blended hydrogel through spinnerets to form monofilaments.

8. The method of claim 6, further comprising:
combining the monofilaments into a thread; and
weaving the thread into a textile.

9. The method of claim 1, further comprising:
adding a composition to the mature mycelium selected from the group consisting of: an enzyme-reactive fluorogenic substrate; an enzyme-reactive chromogenic compound; a nutrient; lavender oil, cannabidiol; an Ayurvedic formulation; a probiotic; an active pharmaceutical ingredient; an amino acid; an antimicrobial agent; a transdermal delivery agent; and any combination thereof.

10. The method of claim 2, further comprising:
adding a composition to the mycelium liquid selected from the group consisting of: an enzyme-reactive fluorogenic substrate; an enzyme-reactive chromogenic compound; a nutrient; lavender oil, cannabidiol; an Ayurvedic formulation; a probiotic; an active pharmaceutical ingredient; an amino acid; an antimicrobial agent; a transdermal delivery agent; and any combination thereof.

11. The method of claim 4, further comprising:
adding a composition to the mycelial hydrogel selected from the group consisting of: an enzyme-reactive fluorogenic substrate; an enzyme-reactive chromogenic compound; a nutrient; lavender oil, cannabidiol; an Ayurvedic formulation; a probiotic; an active pharmaceutical ingredient; an amino acid; an antimicrobial agent; a transdermal delivery agent; and any combination thereof.

12. A mycelium-based product comprising the foam manufactured by the method of claim 1.

13. A mycelium-based product comprising the mycelium liquid manufactured by the method of claim 2.

14. A mycelium-based product comprising the mycelial hydrogel manufactured by the method of claim 4.

15. A mycelium-based product comprising the film manufactured by the method of claim 5.

16. A mycelium-based product comprising the textile manufactured by the method of claim 8.

17. A mycelium-based product manufactured by the method of claim 9, wherein the added composition is the enzyme-reactive fluorogenic substrate or the enzyme-reactive chromogenic compound, and wherein upon exposure to one or more diverse bacterial enzymes, a color of the mycelium-based production visibly changes color and/or the mycelium-based product emits fluorescence.

18. A mycelium-based product manufactured by the method of claim 10, wherein the added composition is the enzyme-reactive fluorogenic substrate or the enzyme-reactive chromogenic compound, and wherein upon exposure to one or more diverse bacterial enzymes, a color of the mycelium-based production visibly changes color and/or the mycelium-based product emits fluorescence.

19. A mycelium-based product manufactured by the method of claim 11, wherein the added composition is the enzyme-reactive fluorogenic substrate or the enzyme-reactive chromogenic compound, and wherein upon exposure to one or more diverse bacterial enzymes, a color of the mycelium-based production visibly changes color and/or the mycelium-based product emits fluorescence.

20. The mycelium-based product manufactured by the method of claim 10, wherein the added composition is gradually released upon contact with skin.
